(19) United States
(12) Patent Application Publication
(10) Pub. No.: US 2023/0310348 A1
(43) Pub. Date: Oct. 5, 2023

(54) USE OF ALVERINE OR ITS DERIVATIVES FOR THE TREATMENT OF MITOCHONDRIAL DISEASES OR DYSFUNCTION ASSOCIATED WITH MITOCHONDRIAL COMPLEX I DEFICIENCIES

(71) Applicants: ASSOCIATION FRANCAISE CONTRE LES MYOPATHIES, Paris (FR); UNIVERSITE D'ANGERS, Angers (FR); CENTRE HOSPITALIER UNIVERSITAIRE D'ANGERS, Angers (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITE PARIS-SACLAY, Saint-Aubin (FR); UNIVERSITE COTE D'AZUR, Nice (FR); UNIVERSITE DE BORDEAUX, Bordeaux (FR)

(72) Inventors: Vincent PROCACCIO, Avrille (FR); Carole SELLEM, Palaiseau (FR); Agnes DELAHODDE, Verrieres-Le-Buisson (FR); Aurelie RENAUD, Angers (FR); Deborah TRIBOUILLARD-TANVIER, Cabanac-Et-Villagrains (FR); Stephane AZOULAY, Nice (FR)

(21) Appl. No.: 18/005,137

(22) PCT Filed: Jul. 26, 2021
(86) PCT No.: PCT/EP2021/070819

§ 371 Date: Jan. 11, 2023

(30) Foreign Application Priority Data
Jul. 24, 2020 (EP) 20305857.3

(51) Int. Cl. A61K 31/137

(52) U.S. Cl. CPC A61K 31/137

(57) ABSTRACT

The present invention relates to the use of Alverine or one of its derivatives for treating diseases associated with mitochondrial dysfunction, especially with mitochondrial complex I deficiency.

### TECHNICAL FIELD

The present invention provides new pharmacological tools for treating mitochondrial diseases or dysfunction, especially those associated with mitochondrial complex I deficiencies.

### STATE OF THE ART

Mitochondrial diseases are chronic, long-term, mostly genetic, often inherited disorders that occur when mitochondria fail to produce enough energy for the body to function properly. Mitochondrial diseases can be present at birth but can also occur at any age. It is estimated that 1 in 4300 people has a mitochondrial disease (Gorman et al., 2015).

Mitochondrial diseases can affect almost any part of the body, including the cells of the brain, nerves, muscles, kidneys, heart, liver, eyes, ears or pancreas. Symptoms of mitochondrial diseases depend on which cells of the body are affected. Patients’ symptoms can range from mild to severe, involve one or more organs, and can occur at any age. Symptoms of mitochondrial diseases can include:
Poor growth
Muscle weakness, muscle pain, low muscle tone, exercise intolerance
Vision and/or hearing problems
Learning disabilities, delays in development, mental retardation
Autism, autism-like features
Heart, cardiac dysfunction, cardiac arrhythmia or conduction defects
Liver or kidney diseases
Gastrointestinal disorders, swallowing difficulties, diarrhea or constipation, unexplained vomiting, cramping, reflux
Diabetes
Increased risk of infection
Neurological problems, seizures, migraines, strokes
Movement disorders
Thyroid and/or adrenal dysfunction
Respiratory (breathing) problems
Lactic acidosis (a buildup of lactate)
Dementia.

Mitochondrial dysfunction can also occur when the mitochondria do not work properly, maybe due to another disease or condition. Many conditions can lead to secondary mitochondrial dysfunction and affect other diseases, including Alzheimer’s or Parkinson diseases, muscular dystrophy, Lou Gehrig’s disease, diabetes and cancer. Individuals with secondary mitochondrial dysfunction do not have primary genetic mitochondrial disease but also suffer from similar symptoms. In addition, some medicines can injure the mitochondria.

Mitochondrial complex I deficiency is the commonest defect seen in more than 30% of mitochondrial diseases. Among them, the two most frequent clinical phenotypes linked to complex I deficiencies are the life-threatening Leigh syndrome or milder phenotypes such as Leber’s optic hereditary neuropathy (LHON). MELAS syndrome has also been considered as a common disorder due to mutations in the mitochondrial genome and associated with a severe reduction of mitochondrial complex I activity.

Complex I is composed of at least 44 subunits, seven of which, i.e. ND1 to 6 and ND4L, are encoded by mitochondrial genes whereas others are encoded by nuclear genes. As a consequence, the clinical and molecular features associated with inherited complex I deficiency are considerably variable. Among those complex I subunits, mutations targeting the NDUFV1 gene have been shown to be responsible for severe neurological phenotypes (Schuelke et al., 1999). Mutations affecting the NDUFS8 subunit have been associated with Leigh syndrome (Procaccio et al., 2004) and mutations targeting the mitochondrial DNA-encoded ND3 subunit were reported in Leigh syndrome or LHON (Sarzi et al., 2007; Wang et al., 2009). Besides, mutations affecting the mitochondrial DNA-encoded ND6 subunit were reported in LHON (Johns et al., 1992).

In addition, complex I deficiency has been identified in secondary mitochondrial dysfunction associated with age related disorders such as Parkinson disease.

Even if most of mitochondrial diseases are of genetic origin, gene therapy seems difficult to implement because of the diversity and complexity of said diseases.

The goal of the present treatments is to improve symptoms and slow progression of the disease or dysfunction with e.g. the following recommendations:
Use vitamin therapy
Conserve energy
Pace activities
Maintain an ambient environmental temperature
Avoid exposure to illness
Ensure adequate nutrition and hydration.

However, there is still a need to find new therapeutical pharmacological approaches for treating said kind of dysfunction or diseases.

### SUMMARY OF THE INVENTION

The inventors have shown that Alverine (ALV), a pharmacological compound mainly known as a smooth muscle relaxant used for functional gastrointestinal disorders, is a potent candidate for treating mitochondrial dysfunction or diseases, especially those associated with complex I deficiencies. The present application reveals that it is efficient for a large spectrum of mitochondrial diseases while displaying low toxicity.

### Definitions

The definitions below represent the meaning generally used in the context of the invention and should be taken into account unless another definition is explicitly stated.

In the frame of the invention, the articles “a” and “an” are used to refer to one or several (i.e., at least one) of the grammatical object of the article. By way of example, “an element” means at least one element, i.e. one or more than one element.

The terms “around”, “about” or “approximately” as used therein when referring to a measurable value such as an amount, a temporal duration and the like should be understood as encompassing variations of ± 20% or ± 10%, preferably ± 5%, more preferably ± 1%, and still more preferably ± 0.1% from the specified value.

Intervals/ranges: throughout this disclosure, various aspects of the invention can be presented in the form of a value interval (range format). It should be understood that the description of values in the form of an interval is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from its natural environment or state. For example, an isolated nucleic acid or peptide is a nucleic acid or peptide which has been extracted from the natural environment in which it is usually found whether this be in a plant or living animal for example. A nucleic acid or peptide for example which is naturally present in a living animal is not an isolated nucleic acid or peptide in the sense of the invention whereas the same nucleic acid or peptide partially or completely separated from other components present in its natural environment is itself “isolated” in the sense of the invention. An isolated nucleic acid or protein can exist in substantially purified form, or can exist in a non-native environment such as, for example, a host cell.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is an animal, preferably a mammal, more preferably a human. It may also be a mouse, a rat, a pig, dog or non-human primate (NHP), such as the macaque monkey.

In the sense of the invention, a “disease” or “pathology” is a state of health of an animal in which its homeostasis is adversely affected and which, if the disease is not treated, continues to deteriorate. Conversely, in the sense of the invention, a “disorder” or “dysfunction” is a state of health in which the animal is able to maintain homeostasis but in which the state of health of the animal is less favourable than it would be in the absence of the disorder. Left untreated, a disorder does not necessarily result in deterioration in the state of health of the animal over time.

A disease or disorder is “alleviated” (“reduced”) or “ameliorated” (“improved”) if the severity of a symptom of the disease or disorder, the frequency with which such a symptom is experienced by the subject, or both of these, is reduced. This also includes the disappearance of progression of the disease, i.e. halting progression of the disease or disorder. A disease or disorder is “cured” (“recovered”) if the severity of a symptom of the disease or disorder, the frequency with which such a symptom is experienced by the patient, or both, is eliminated.

In the context of the invention, a “therapeutic” treatment is a treatment administered to a subject who displays the symptoms (signs) of pathology, with the purpose of reducing or removing these symptoms. As used herein, the “treatment of a disease or disorder” means reducing the frequency or severity of at least one sign or symptom of a disease or disorder experienced by the subject. A treatment is said to be prophylactic when it is administered to prevent the development, spread or worsening of a disease, particularly if the subject does not have or does not yet have the symptoms of the disease and/or for which the disease has not been diagnosed.

As used herein, “treating a disease or disorder” means reducing the frequency or severity of at least one sign or symptom of a disease or disorder experienced by a subject. Disease and disorder are used interchangeably herein in the context of treatment.

In the sense of the invention, an “effective quantity” or an “effective amount” of a compound is that amount of compound which is sufficient to provide a beneficial effect to the subject to which the compound is administered. The expression “therapeutically effective quantity” or “therapeutically effective amount” refers to a quantity which is sufficient or effective to prevent or treat (in other words delay or prevent the development, prevent the progression, inhibit, decrease or reverse) a disease or a disorder, including alleviating symptoms of this disease or disorder.

### DETAILED DESCRIPTION OF THE INVENTION

The present invention relates to the use of Alverine (ALV) or one of its derivatives, e.g. 4-hydroxy Alverine which is the first metabolite in the Alverine degradation pathway, for treating a disease associated with mitochondrial dysfunction.

More specifically and according to a first aspect, the present invention thus relates to a pharmaceutical composition comprising at least Alverine (ALV) or one of its derivatives, e.g. 4-hydroxy Alverine, for use in the treatment of a disease associated with mitochondrial dysfunction.

In other words, a composition comprising Alverine (ALV) or one of its derivatives, e.g. 4-hydroxy Alverine, is used to prepare a medicament intended for the treatment of a disease associated with mitochondrial dysfunction.

The invention thus relates to a method of treating a disease associated with mitochondrial dysfunction, comprising administering to a subject in need thereof, at an efficient dose, a composition comprising Alverine (ALV) or one of its derivatives, e.g. 4-hydroxy Alverine.

Alverine (noted ALV), also named N-ethyl-3-phenyl-N-(3-phenylpropyl)propan-1-amine or Spasmaverine or Dipropyline or Sestron or Phenpropamine or Alverina or N-ethyl-3,3′-diphenyldipropylamine or Alverinum or Phenopropamine or Profenil, is a tertiary amine having one ethyl and two 3-phenylprop-1-yl groups attached to the nitrogen. It has the CAS number 150-59-4 and the following formula:

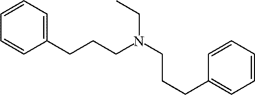 [SMILES: CCN(CCCc1ccccc1)CCCc1ccccc1]

It is generally in the form of a white powder having high solubility, in e.g. alcohol or chloroform.

Alverine is a drug used as a smooth muscle relaxant to support the treatment of functional gastrointestinal dysmotility. It is for example sold under the trade names DOLOSPASMYL or METEOSPASMYL that correspond to capsules containing 60 mg thereof. Alverine is in the form of its citrate salt and formulated with simethicone. In that context, the recommended daily dose is 2 or 3 capsules, i.e. 120 or 180 mg.

Also encompassed by the present invention are derivatives of Alverine, having the same biological activity, especially as reported in the examples, e.g. on mitochondrial complex I activity or mitochondrial respiration.

The term “Alverine derivatives” encompasses derivatives and metabolites as well as esters and pharmaceutically acceptable salts for the preparation of pharmaceutical compositions. A derivative is a compound originating from another (the precursor, with a typically similar chemical structure) after transformation of the latter. The derivative may differ from one or more atoms or functional groups. A metabolite is an intermediate stable compound or a compound resulting from the biochemical transformation of an initial molecule by metabolism.

According to the present invention, an “Alverine derivative” means, inter alia, the mono- or poly-hydroxylated derivatives on the phenyl nuclei and the mono- or poly-hydroxylated or mono- or poly-carboxylated rings on the aliphatic chains.

The term “pharmaceutically acceptable salts” means the addition salts of Alverine, which can be obtained by reaction of this compound with a mineral or organic acid according to a method known per se. Among the acids which can be used for this purpose are hydrochloric, hydrobromic, sulfuric, phosphoric, 4-toluene sulfonic, methane sulfonic, cyclohexyl sulfamic, oxalic, succinic, formic, fumaric, maleic, citric, aspartic, cinnamic, lactic, glutamic acids. N-acetyl-aspartic, N-acetyl-glutamic, ascorbic, malic, benzoic, nicotinic and acetic, citrate and Alverine tartar have been widely used in pharmaceutical spasmolytic preparations.

Among the esters on the hydroxy function, mention may be made of carboxylic acid esters having from 1 to 6 carbon atoms.

Examples of such derivatives include:
Alverine citrate (CAS number: 5560-59-8) of formula:

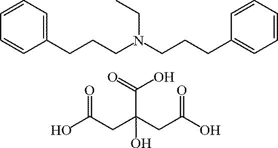

Alverine-d5 citrate (CAS number: 1215327-00-6) of formula:

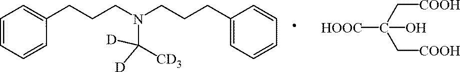

4-hydroxy Alverine (or para hydroxy alverine; CAS number: 142047-94-7) or 4-hydroxy Alverine HCl (hydrochloride salt) of formula:

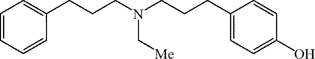 [SMILES: CCN(CCCc1ccccc1)CCCc1ccc(O)cc1]

4-hydroxy Alverine-d5 (CAS number: 1216415-67-6) of formula:

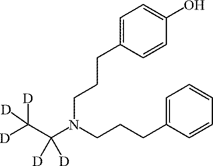 [SMILES: [2H]C([2H])([2H])C([2H])([2H])N(CCCc1ccccc1)CCCc1ccc(O)cc1]

4-hydroxy Alverine Glucuronide of formula:

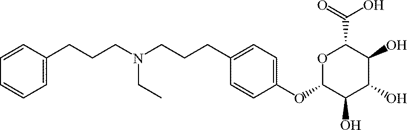 [SMILES: CCN(CCCc1ccccc1)CCCc1ccc(O[C@@H]2O[C@H](C(=O)O)[C@@H](O)[C@H](O)[C@H]2O)cc1]

N-Desethyl Alverine HCl (CAS number: 93948-20-0) of formula:

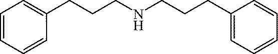 [SMILES: c1ccc(CCCNCCCc2ccccc2)cc1]

Of particular interest are Alverine citrate and 4-hydroxy Alverine.

Said compounds, including Alverine, can be further modified to increase their stability, their bioavailability and/or their ability to reach the target tissues, especially mitochondria.

As known by the skilled person, said compounds, especially Alverine, may be present in the composition in a naked form (free) or contained in delivery systems, which increase the stability, the targeting and/or the biodisponibility, such as liposomes, or incorporated into carriers such as hydrogels, cyclodextrins, biodegradable nanocapsules, bioadhesive microspheres, vectors or in combination with a cationic peptide.

The present invention also concerns pharmaceutical compositions containing as an active ingredient at least a compound as defined above, as well as the use of this compound or composition as a medicinal product or medicament.

According to a specific embodiment and especially in relation to Alverine citrate, a pharmaceutical composition according to the invention may comprise simethicone. According to an alternative embodiment, such a composition is deprived of simethicone.

The present invention then provides pharmaceutical compositions comprising a compound according to the invention. Advantageously, such compositions comprise a therapeutically effective amount of said compound, and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington’s Pharmaceutical Sciences” by E. W. Martin. Such compositions will contain a therapeutically effective amount of the therapeutic, preferably in purified form, together with a suitable amount of carrier so as to provide the form for proper administration to the subject.

In a preferred embodiment, the composition is formulated in accordance with routine procedures as a pharmaceutical composition adapted for e.g. oral administration to human beings. Typically, compositions for oral administration are in the form of capsules or tablets, possibly scored tablets or effervescent tablets, further containing excipients suitable for solid dosage form and administration in humans. As an example, available commercial forms of Alverine are capsules of Alverine citrate, which further contain simethicone, gelatin, glycerol and titanium dioxide.

Alternatively, the composition may be in a liquid form, advantageously an aqueous composition. Any other suitable solvent can be used.

The amount of the therapeutic agent of the invention, i.e. a compound as disclosed above, which will be effective in the treatment of a disease can be determined by standard clinical techniques. In addition, in vivo and/or in vitro assays may optionally be employed to help predict optimal dosage ranges. The precise dose to be employed in the formulation will also depend on the route of administration, the weight and the seriousness of the disease, and should be decided according to the judgment of the practitioner and each patient’s circumstances.

According to a specific embodiment, the composition of the invention is in a solid form, advantageously a capsule or tablet, more advantageously comprising 60 mg of the active compound, or even less. Preferably, the composition comprises a quantity equal to or less than 50 mg, 40 mg, 30 mg, 20 mg or even equal to or less than 10 mg or 5 mg.

According to another embodiment, the composition of the invention is in a liquid form and advantageously comprises less than 10 µM of the active compound, or even less than 3 µM, advantageously 1 µM or less, more advantageously between 100 nM and 300 nM.

Suitable administration should allow the delivery of a therapeutically effective amount of the therapeutic product to the target tissues, depending on the disease.

Alverine or its derivatives can be administered in a pharmaceutically acceptable form by one of the various routes known for this type of active principle.

Available routes of administration are topical (local), rectal, enteral (system-wide effect, but delivered through the gastrointestinal (GI) tract), intranasal or parenteral (systemic action, but delivered by routes other than the GI tract). In the specific case of mitochondrial diseases, the preferred route of administration of the compositions disclosed herein is generally enteral which includes oral, sublingual, buccal administration, preferably oral administration.

According to other embodiments, it can be a parenteral administration, especially via intramuscular (i.e. into the muscle) or systemic administration (i.e. into the circulating system). In this context, the term “injection” (or “perfusion” or “infusion”) encompasses intravascular, in particular intravenous (IV), intraperitoneal (IP) and intramuscular (IM) administration. Injections are usually performed using syringes or catheters.

According to one embodiment, the composition is administered orally, intramuscularly, intraperitoneally, subcutaneously, topically, locally or intravascularly.

The pharmaceutical composition according to the invention can be in any of the usual oral dosage forms comprising tablets, capsules and liquid preparations such as elixirs and suspensions containing various masking substances for coloring, flavor and stabilization.

According to a preferred embodiment, the composition is for oral administration. Advantageously, the composition is administered per os, i.e. by way of the mouth.

Preferably, a composition according to the invention is administered orally, in particular in the form of capsules or tablets.

To make the oral dosage forms according to the invention, in particular capsules, the active substance can be mixed with various conventional materials such as starch, calcium carbonate, lactose, sucrose and dicalcium phosphate to facilitate the process of encapsulation. Magnesium stearate, as an additive, provides a useful lubricant function if necessary.

It may in certain cases be advantageous to provide forms with controlled release, in particular sustained release by known galenical forms.

Likewise, a composition according to the invention is for the preparation of a pharmaceutical composition which can be administered by injectable route.

The pharmaceutical composition according to the invention can be dissolved or suspended in a sterile injectable liquid pharmaceutically acceptable, such as sterile water, a sterile organic solvent or a mixture of these two liquids for intravenous administration.

Other routes of administration may include, but are not limited to, subcutaneous implants, as well as oral, sublingual, transdermal, topical, intranasal or rectal administration. Biodegradable and non-biodegradable delivery systems can also be used.

As already mentioned, a composition according to the invention is preferably in a solid dosage form adapted for oral administration, advantageously in the form of one or more capsules or tablets. Thus, they can be taken with a little water before or during the main meal.

According to a preferred embodiment, the composition according to the invention is administered daily, for example once per day, even twice, or even three times per day. The treatment can last several weeks, several months, several years or even for the whole life.

In general, the dosage of therapeutic agent, i.e. Alverine or one of its derivatives, will vary depending upon such factors as the subject’s age, weight, height, gender, general medical condition and previous medical history. Typically, it is desirable to provide the patient with an individual dose of the therapeutic agent, which is efficient without being toxic.

According to a particular embodiment of the invention, the dosage of the composition, advantageously the daily dosage to be taken orally by a human, is inferior or equal to 10 mg/kg or 9, 8, 7, 6, 5, 4, 3 mg/kg, or even inferior or equal to 2.5, 2, 1.5 or 1 mg/kg, or even inferior or equal to 0.9, 0.8, 0.7, 0.6, 0.5, 0.4, 0.3 or 0.1 mg/kg.

As already stated, the patient is advantageously a human, particularly a newborn, a young child, a child, an adolescent or an adult. The therapeutic tool according to the invention, however, may be adapted and useful for the treatment of other animals, particularly mice, pigs, dogs or macaque monkeys.

As already mentioned, the present invention relates to the treatment of mitochondrial diseases in general, i.e. diseases linked to or caused by mitochondrial dysfunction. In the frame of the present application, the wording “a disease associated with mitochondrial dysfunction” is used in order to encompass all these situations.

In relation to the examples showing a positive effect of Alverine or one of its derivatives on the mitochondrial respiratory chain, diseases of particular interest are mitochondrial respiratory chain diseases.

Several mitochondrial diseases have been well documented in the prior art:

NARP (neuropathy, ataxia, and retinitis pigmentosa) syndrome is caused by various mutations in the mitochondrially-encoded ATP6 gene, which encodes a subunit of ATPase (OXPHOS complex V). The mutations are often heteroplasmic (co-existence of both mutant and wt mitochondrial DNA, mtDNA) within the same cells. Depending both on the type of mutation and on the percentage of mutant mtDNA (degree of heteroplasmy), the clinical outcomes are more or less severe. The ATP6 m.8993T>C/G mutations are among the most frequent in NARP patients and lead to severe forms of the NARP syndrome. FMC1 is a nuclear gene that encodes a protein required at high temperature (35-37° C.) for assembly of the F1 sector of ATP synthase, thereby mimicking the heteroplasmy observed in NARP patients. Indeed, when grown at restrictive temperature (35-37° C.), the mitochondria of the fmc1Δ mutant contain far fewer assembled ATP synthase complexes than a wild-type (WT) strain but the ones that assemble are fully functional. This heterogeneity is also found in patients with decreased levels of ATP synthase due to heteroplasmic ATP6 mutations. Therefore, the fmc1Δ mutant constitutes an appropriate model of these disorders, in particular the equivalent of m.8993T>G (MR14, NARP) mutant (Schon, E.A. et al., 2001).

The TAZ gene encodes tafazzin, a mitochondrial transacylase that catalyzes remodeling of immature cardiolipin to its mature composition containing a predominance of tetralinoleoyl moieties. TAZ mutations result in Barth syndrome, an X-linked disease conventionally characterized by dilated cardiomyopathy (CMD) with endocardial fibroelastosis (EFE), a predominantly proximal skeletal myopathy, growth retardation, neutropenia, and organic aciduria, particularly excess of 3-methylglutaconic acid (Barth, P.G. et al., 1996).

SHY1 is a yeast homolog of the human SURF1 gene. The SURF1 gene encodes an assembly factor of mitochondrial complex IV. SURF1 mutations are associated with Leigh syndrome, a progressive and severe neurodegenerative disorder with onset within the first months or years of life, and may result in early death. Affected individuals usually show global developmental delay or developmental regression, hypotonia, ataxia, dystonia, and ophthalmologic abnormalities, such as nystagmus or optic atrophy.

SYM1 is a yeast homolog of the human MPV17 gene. MPV17 encodes a mitochondrial inner membrane protein of unknown function. MPV17 mutations cause mitochondrial DNA depletion syndrome, an autosomal recessive disorder characterized by infantile onset of progressive liver failure, often leading to death in the first year of life. Those that survive develop progressive neurologic involvement, including ataxia, hypotonia, dystonia, and psychomotor regression (Spinazzola, A. et al., 2006).

According to a specific embodiment, the diseases to be treated in the frame of the invention are linked to or due to at least one gene defect in at least one of the following genes: MTTL1, ATP6, TAZ, SURF1, POLG, MPV17, OPA1, COA6, ND6 and BCS1L.

Of particular interest is the treatment of a disease selected in the group consisting of: MELAS syndrome, maternally inherited myopathy and cardiomyopathy, NARP syndrome, Leigh syndrome, Barth syndrome, Mitochondrial DNA Depletion Syndrome 4A (Alpers Type), Mitochondrial DNA Depletion Syndrome 4B (MNGIE Type), Mitochondrial recessive ataxia syndrome, Sensory Ataxic Neuropathy Dysarthria and Ophthalmoplegia, Spinocerebellar Ataxia with Epilepsy, Progressive External Ophthalmoplegia, Mitochondrial DNA depletion syndrome-6, Navajo neuropathy, Behr Syndrome, Mitochondrial DNA Depletion Syndrome 14, infantile cardioencephalomyopathy due to cytochrome c oxidase deficiency (COA6 mutations), Mitochondrial Complex III Deficiency Nuclear Type 1, GRACILE Syndrome and Bjornstad Syndrome.

Of particular interest is the treatment of diseases associated with mitochondrial complex I deficiency/deficiencies. Some diseases are merely linked to a dysfunction of Complex I, whereas other diseases are associated with multiple deficits, for example in several mitochondrial complexes.

As known in the art, the respiratory chain in mitochondria is the base of the oxidative phosphorylation, which is an important cellular process that uses oxygen and simple sugars to create adenosine triphosphate (ATP), the cell’s main energy source. Five protein complexes, made up of several proteins each, are involved in this process. The complexes are named complex I, complex II, complex III, complex IV, and complex V. The complex I (CI or NADH dehydrogenase or NADH coenzyme Q reductase), the first enzyme in the respiratory chain, is a very large protein complex (around 1000 kDa) composed of at least 44 subunits including 7 encoded by mitochondrial DNA (ND1 to ND6 and ND4L).

According to a specific embodiment, Alverine or one of its derivatives can be used to treat a so-called “primary” mitochondrial disease, i.e. due to a genetic abnormality identified in at least one subunit of the complex I linked to either pathogenic mitochondrial or nuclear DNA mutation(s). These pathologies are associated with neurological, cardiac muscular or ophthalmological symptoms, which are the most affected tissues or organs in such mitochondrial diseases even if other organs or tissues are possibly affected.

According to another particular embodiment, Alverine or one of its derivatives can be used to treat a so-called “secondary” mitochondrial disease. In that case, the genetic anomaly does not directly imply complex I but the pathology will affect mitochondrial functions and in particular may lead to a reduction in the enzymatic activity of this complex I. Such a disease may also be due to nongenetic causes such as environmental factors or ageing. This is the case especially in Parkinson disease or other age-related neurodegenerative disorders.

According to one embodiment, mitochondrial deficiency or dysfunction results from a genetic disease.

Genetic diseases are, by definition, diseases resulting from one or a plurality of gene defects (or mutations) in one or a plurality of genes. The gene defects can affect mitochondrial DNA and/or nuclear genes. The gene defects responsible for the mitochondrial diseases may be point mutations, leading to a codon change. However, the diseases may be linked to the deletion or insertion of one or more bases or codons.

According to a specific embodiment, the disease results from one or a plurality of gene defects (or mutations) in one or a plurality of genes involved in complex I functionality.

A non-limiting list of such genes includes:
complex I structural genes, especially MTND1 (or ND1), MTND2 (or ND2), MTND3 (or ND3), MTND4 (or ND4), MTND5 (or ND5), MTND6 (or ND6), MTND4L (or ND4L), NDUFA1, NDUFA2, NDUFA3, NDUFA4, NDUFA5, NDUFA6, NDUFA7, NDUFA8, NDUFA9, NDUFA10, NDUFA11, NDUFA12, NDUFA13, NDUFAB1, NDUFB1, NDUFB2, NDUFB3, NDUFB4, NDUFB5, NDUFB6, NDUFB7, NDUFB8, NDUFB9, NDUFB10, NDUFB11, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3;
complex I assembly genes, especially NDUFAF1, NDUFAF2, NDUFAF3, NDUFAF4, NDUFAF5, NDUFAF6, NDUFAF7, NDUFAF8, NUBPL, ACAD9, TMEM126B, FOXRED1, ECSIT, AIF, TIMMDC1.

As an example, Alverine or one of its derivatives can be used to treat a genetic disease wherein the genetic defect concerns ND3, ND6, NDUFV1 or NDUSF8.

Exemplary mutations are the NDUFV1 mutations c.1162+2A>C and c.1156C>T resulting in p.Arg386Cys amino change substitution or the NDUFV1 mutation resulting in p.Ala341val amino change substitution, mostly responsible for neurological disorders or Leigh syndrome.

Several mitochondrial diseases of genetic origin, especially in relation to mitochondrial complex I deficiencies, have been well documented in the prior art:

LHON syndrome or Leber Hereditary Optic Neuropathy usually begins in young adults. The onset is abrupt with a rapid drop in vision in the center of the eye, corresponding to a decrease in central visual acuity. Most often, a peripheral visual field persists, much like a halo of vision around a blind area. This disease is due to homoplasmic mutations in genes encoding respiratory chain complex I subunits. In practice, the following mitochondrial DNA mutations m.11778G> A, m.3460G> A and m.14484T> C represent about 95% of LHON mutations.

Leigh syndrome (or LS) is a progressive and severe neurodegenerative disorder. Affected individuals usually show global developmental delay or developmental regression, hypotonia, ataxia, dystonia, and ophthalmologic abnormalities, such as nystagmus or optic atrophy. Leigh syndrome can also have detrimental multisystemic effects on the cardiac, hepatic, gastrointestinal, and renal organs. Biochemical studies in patients with Leigh syndrome tend to show increased lactate and abnormalities of mitochondrial oxidative phosphorylation. Leigh Syndrome may be associated with mutations in genes encoding complex I subunits such as NDUFV1 mutations or the MTND5 m.13513G>A mutation.

MELAS syndrome, comprising Mitochondrial myopathy, Encephalopathy, Lactic Acidosis, and Stroke-like episodes, is a genetically heterogeneous mitochondrial disorder with a variable clinical phenotype. The disorder is accompanied by features of central nervous system involvement, including seizures, hemiparesis, hemianopsia, cortical blindness, and episodic vomiting. This syndrome was first associated to the m.3243A>G mutation in mitochondrial DNA, i.e. in the tRNA^{Leu} ^{(UUR)} (MTTL1) gene, which induces an alteration in the translation of complex I mRNA into proteins and therefore, a reduction in the quantity of structural proteins of complex I such as ND6. MELAS syndrome can also be associated with other mitochondrial DNA mutations such as the m.3260A>G mutation, which also affects tRNA^{Leu} ^{(UUR)}. This m.3260A>G mutation may also result in other clinical phenotypes including maternally inherited myopathy and cardiomyopathy.

Of particular interest is the treatment of a genetic disease shown to be associated with complex I deficiency, e.g. MELAS syndrome, Leigh syndrome and Leber Hereditary Optic Neuropathy. It is to be noted that such diseases may be associated with other symptoms such as cardiac, myopathy or neurological clinical phenotypes.

More generally, Alverine or one of its derivatives can be used to treat mitochondrial dysfunction, especially mitochondrial dysfunction associated with complex I deficiencies. Mitochondrial dysfunction, characterized by a loss of efficiency in the electron transport chain and reductions in the synthesis of high-energy molecules such as adenosine-5′-triphosphate (ATP), is a characteristic of aging and essentially of all chronic diseases.

These diseases include:
neurodegenerative diseases, such as Alzheimer’s disease, Parkinson’s disease, Huntington’s disease, amyotrophic lateral sclerosis (Lou Gehrig’s disease), and Friedreich’s ataxia;
cardiovascular diseases, such as atherosclerosis and other heart and vascular conditions;
diabetes and metabolic syndrome;
autoimmune diseases, such as multiple sclerosis, systemic lupus erythematosus, and type 1 diabetes;
neurobehavioral and psychiatric diseases, such as autism spectrum disorders, schizophrenia, and bipolar and mood disorders;
gastrointestinal disorders;
fatiguing illnesses, such as chronic fatigue syndrome and Gulf War illnesses;
musculoskeletal diseases, such as fibromyalgia and skeletal muscle hypertrophy/atrophy;
muscular dystrophies;
cancer; and
chronic infections.

According to a specific embodiment, functional gastrointestinal dysmotility is out of the definition of the diseases to be treated in the frame of the present invention.

According to one aspect, the composition according to the invention is associated with at least another compound for the treatment of the same disease. The composition according to the invention and the said compound can be administered simultaneously or separately over time to take into account their particularities and in particular their bioavailability.

According to a specific embodiment, the present invention concerns a composition, advantageously a pharmaceutical composition or a medicinal product containing a compound as described above and potentially other active molecules (other gene therapy proteins, chemical groups, peptides or proteins, etc.) for the treatment of the same disease or a different disease, advantageously of the same disease.

Preferably, the pharmaceutical composition according to the present invention and at least one compound for the treatment of same or different disease are administered simultaneously, separately or spread over time to treat the same or different disease.

More generally, in relation to mitochondrial diseases, a further compound able to ameliorate mitochondrial function can be administered simultaneously or at different times. In case of simultaneous administration, the two compounds can be associated in the same composition.

Examples of such further compounds are natural supplements, such as L-carnitine, alpha-lipoic acid (α-lipoic acid [1,2-dithiolane-3-pentanoic acid]), coenzyme Q10 (CoQ10 or ubiquinone), riboflavin (B2 vitamin) reduced nicotinamide adenine dinucleotide (NADH), L-arginine, possibly in combination.

Examples of compounds used e.g. in the case of MELAS syndrome are Nitric Oxide (NO) precursors such as L-arginine and citrulline.

Subjects that could benefit from the compositions of the invention include all patients having a disease associated with mitochondrial dysfunction, especially a mitochondrial dysfunction associated with complex I deficiencies, diagnosed with such a disease or at risk of developing such a disease.

A subject to be treated with a composition according to the invention can be selected based on various criteria. In relation to mitochondrial dysfunction, in particular complex I deficiencies, several tests can be performed, e.g.:
At the biochemical level: based on a biopsy, especially muscle or skin biopsies of the subject, the O2 consumption and/or the mitochondrial complex I activity (as disclosed in examples 8 to 9 below) can be measured. The activity of the other complexes of the respiratory chain can be further evaluated to determine if the mitochondrial dysfunction is only due to complex I deficiencies;
At the genetic level: sequencing DNA extracted from blood, cells or a biopsy sample, for example of skin, makes it possible to identify one or more molecular abnormality, especially mutations or deletions/insertions in the preferred genes listed above. Alternatively, the corresponding protein expression or activity can be evaluated by any method known to the one skilled in the art (e.g. western blotting).

A target of the invention is to provide a safe (not toxic) treatment. A further aim is to provide an efficient treatment which allows to postpone, slow down or prevent the development of the disease, and possibly to ameliorate the phenotype of the patient which can be easily monitored at the clinical level as disclosed below.

In a subject, the composition according to the invention can be used:
for ameliorating mitochondrial function, especially mitochondrial respiration;
for ameliorating growth;
for ameliorating muscle function;
for ameliorating vision and/or hearing;
for ameliorating respiratory function;
for ameliorating heart, liver or kidney function;
for ameliorating brain function;
for ameliorating digestive function; and/or
for prolonging survival, more generally to ameliorate the quality and the expectancy of life.

According to one aspect, the invention concerns a method for ameliorating mitochondrial function, especially complex I activity, advantageously without adverse effects, comprising administering to a subject in need thereof a therapeutic quantity of a composition as disclosed above.

Advantageously, said ameliorations are observed for up to 1 month after starting the treatment, or 3 months or 6 months or 9 months, more advantageously for up to 1 year after starting the treatment, 2 years, 5 years, 10 years, or even for the whole life of the subject.

In one embodiment, said ameliorations result in reduced symptom severity and/or frequency and/or delayed appearance.

An amelioration can be evaluated based on methods known in the art, e.g. in the case of MELAS:
assessment of the lactate level, especially cerebral ventricular lactate, as measured e.g. by Magnetic Resonance Spectroscopy (MRS);
assessment of quality and/or expectancy of life by clinical scales, e.g. NMDAS (Newcastle Mitochondrial Disease Scale for Adults) score or SF-36 (Short Form Health Survey) score;
assessment of the changes in brain e.g. using Magnetic Resonance Imaging (MRI);
assessment of the changes in muscle activity using physical tests such as the six minute Walk Test;
assessment of the changes in venous lactate and GDF 15 concentration;
assessment of the changes in mtDNA heteroplasmy in urine and blood.

The adequate parameters for a given case can be adapted depending on the disease.

Thus, the claimed treatment allows improving the clinical state and the various parameters disclosed above in comparison with an untreated subject.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction/ Principles, Applications and Troubleshooting” (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

Without further description, it is believed that one of ordinary skill in the art can, using the preceding description and the following illustrative examples, make and utilize the compounds of the present invention and practice the claimed methods.

### EXAMPLES

The invention and its advantages are understood better from the examples shown below supporting the annexed figures. In particular, the present invention is illustrated with regard to the effect of Alverine citrate (ALV) on various model organisms for mitochondrial diseases as well as on patient cell lines.

ALV has been shown to be active on several complex I subunits of different organisms carrying mutations in the following subunits: NDUFV1 (Schuelke et al., 1999), NDUFS8 (Procaccio et al., 2004), ND2 or ND3 (Sarzi et al., 2007), and ND6 (Johns et al., 1992) subunits.

In addition, 4-hydroxy Alverine (4-Hydroxy ALV) a metabolite of ALV was shown to be active on Podospora anserina nuo-51 gene carrying the mutation A357V.

Besides, the efficacy of Alverine has been demonstrated on other mitochondrial yeast mutants. These examples are not however in any way limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Effect of the thermosensitive mutation A357V in the Podospora anserina nuo-51 gene, mimicking the pathogenic NDUFV1 A341V mutant in human, on the growth rate.

FIG. 2: Effect of the thermosensitive mutation A357V in the Podospora anserina nuo-51 gene, mimicking the pathogenic NDUFV1 A341V mutant in human, on respiration (O2) and complex I activity (CI) at 31.5° C. CS: Citrate synthase activity. The asterisks (** or ***) indicate a statistically significant difference versus the wild type strain (WT).

FIG. 3: Effect of ALV (A) and 4-Hydroxy ALV (B) in comparison to DMSO (C) on the thermosensitive growth at 33° C. of the nuo-51 ^{A357V} mutant of Podospora anserina mimicking the human pathogenic mutation NDUFV1^{A341V}.

FIG. 4: Effect of various concentrations of ALV (0.001 to 10 µM; VEH = 0) on the growth rate of the nuo-51^{A357V} (NDUFV1) at 31.5° C., the Δnuo-19 (NDUFS7) and the nd2-nd3 (ND) mutant strains at 28° C. The asterisks (* or **) indicate a statistically significant difference versus the untreated cultures (VEH = vehicle).

FIG. 5: Effect of ALV 1 µM on the O2 consumption rate of nuo-51^{A357V} (NDUFV1) at 31.5° C. The asterisks (**) indicate a statistically significant difference versus the untreated cultures (VEH = vehicle).

FIG. 6: Effect of ALV 1 µM on complex I activity (CI) of nuo-51^{A357V} (NDUFV1) at 31.5° C. The asterisks (***) indicate a statistically significant difference versus the untreated cultures (VEH = vehicle). CS: Citrate synthase activity.

FIG. 7: Effect of ALV 1 µM on the C. elegans nuo-1^{A352V} (NDUFV1) mutant progeny. The asterisks (**) indicate a statistically significant difference versus the untreated cultures.

FIG. 8: Effect of ALV 1 µM on the progeny of wild-type worms subjected to NDUFV1 or NDUFS8 RNAi. The asterisks (**) indicate a statistically significant difference versus the untreated cultures.

FIG. 9: Determination of the maximal ALV concentration nontoxic for the growth of NDUFV1 mutant cells carrying the compound heterozygous mutations c1162+2A>C and c.1156C>T (p.Arg386Cys): range of ALV concentrations from 30 nM to 30 µM.

FIG. 10: Determination of the active ALV concentration on citrate synthase activity of NDUFV1 mutant cells carrying the compound heterozygous mutations c.1162+2A>C and c.1156C>T (p.Arg386Cys). The asterisks (*) indicate a statistically significant difference versus the untreated cells (Vehicle).

FIG. 11: Determination of the active ALV concentration on complex I enzyme activity of NDUFV1 mutant cells carrying the compound heterozygous mutations c.1162+2A>C and c.1156C>T (p.Arg386Cys). The asterisks (* or ** or ***) indicate a statistically significant difference versus the untreated cells (Vehicle).

FIG. 12: Determination of the active ALV concentration on complex I enzyme activity (CI) normalized with respect to citrate synthase activity (CS) of NDUFV1 mutant cells carrying the compound heterozygous mutations c.1162+2A>C and c.1156C>T (p.Arg386Cys). The asterisks (* or **) indicate a statistically significant difference versus the untreated cells (Vehicle).

FIG. 13: Determination of the maximal ALV concentration nontoxic for the growth of ND6 mutant cells carrying the m.14484T>C (p.Met64Val) mitochondrial DNA mutation: range of ALV concentrations from 30 nM to 10 µM. Veh: Vehicule. n =4 +/- SEM. * P<0.05.

FIG. 14: Measurement of complex I enzyme activity of ND6 mutant in the presence of Alv. (A): after 48 h treatment with different concentrations of Alv, normalized by citrate synthase for ND6 cell line. (B): Enzymatic measurement of citrate synthase activity for ND6 mutant cell line. n =4 +/- SEM. * P<0.05.

FIG. 15: Determination of lactate production for control fibroblast cells and NDUFV1 and ND6 mutant fibroblast cell lines after 48h exposure with Alv (A), Control cells; NDUFV1 (B) and ND6 (C). (A) et (B): n = 3. (C): n=4 +/- SEM. * P<0.05

FIG. 16: Effect of Alverine on growth of various mutant yeast strains on a solid respiratory medium as detected by halo tests.

FIG. 17: Determination of the active range of concentration of ALV leading to suppression of the respiratory growth defect of the taz1 mutant yeast strain.

### EXAMPLES 1 TO 5: PODOSPORA ANSERINA MODELS

The filamentous fungus Podospora anserina strain used in examples 1 to 5 contains a specific mutation modeling a human mutation in the NDUFV1 subunit of the complex I resulting in a mitochondrial disease. This strain presents a growth defect at the non-permissive temperature more than 31.5° C.

P. anserina mutant strains:
nuo-51 ^{A357V} → The A357V mutation was introduced in the nuo-51 gene of the S wild type strain in association with the nourseothricin resistant cassette (Nat^{R}). In order to fully mimic the human NDUFV1^{A341V} disease the NDI-1 and AOX genes were inactivated (El-Khoury et al., 2008).
   Genotype of the strain: S, mat⁻, nuo-51 ^{A357V}, Δndi-1, Δaox, Nat^{R}, Hygro^{R}
nd2-nd3 → The Wa32-LL strain carries a mitochondrial plasmid integrated into the 5′-UTR of the mitochondrial co-transcriptional unit nd2/nd3 down regulating the level of the corresponding subunits ND2 and ND3 (Maas et al., 2007)
Δnuo-19 → The gene encoding the homolog of the human PSST (NDUFS7) subunit was inactivated (Maas et al., 2010) in the S wild type strain.

### Example 1: Effect of the Thermosensitive Mutation A357V in the Podospora Anserina Nuo-51 Gene Mimicking the Pathogenic Mutation NDUFV1^{A341V} in Human on the Growth Rate (1), the Respiration and Complex I Activity (2)

### Materials and Methods

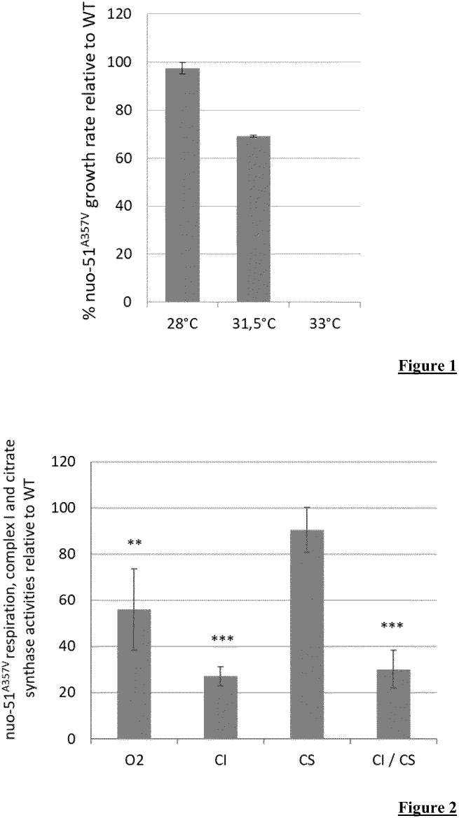

The mycelium is grown on solid synthetic M2 media containing Petri dishes at 28° C. initiated from ascospores. Cultures are propagated on solid synthetic medium (M2, http://podospora.i2bc.paris-saclay.fr/) from small pieces of mycelium. Growth rates are measured in centimeters of mycelial growth per day. In FIG. 1, growth rates are expressed relative to the wild type.

For oxygen consumption, 40 mg samples of mycelium grown at 31.5° C. are introduced in an Oxytherm apparatus (Hansatech electrode). After a 2 min registration, samples are recovered, and protein content estimated with a Bradford protein assay (Biorad). Respirations are registered as nmole of oxygen consumed/min/µg protein and then expressed relative to the wild type. At least 3 samples for each strain were examined per experiment. The experiment was repeated at least 5 times.

Complex I activity (CI) was performed on crude mitochondria obtained after mixing the mycelium with glass beads in 0.4 M sucrose buffer and differential centrifugations. The rates of NADH consumption in presence and absence of rotenone was registered by spectrophotometry (340-380 nm) with 25 µg of mitochondria starting with NADH and quinone addition. Citrate synthase activity (CS) was determined on equivalent samples of mitochondria to express the complex I activity as the NADH dehydrogenase – rotenone sensitive activity relative to citrate synthase activity (CI/CS).

### Results

The results are shown in FIGS. 1 and 2.

The nuo-51^{A357V} strain is thermosensitive compared to the wild-type strain with a 30% reduction of growth rate at 31.5° C. and an absence of growth at 33° C. (FIG. 1).

The nuo-51^{A357V} mutant strain exhibits a 40% reduction in oxygen consumption and a 70% decrease in complex I activity (FIG. 2).

### Example 2: Effect of ALV and 4-Hydroxy ALV on the Thermosensitive Growth of the Nuo-51 ^{A357V} Mutant of Podospora Anserina Mimicking the Human Pathogenic Mutations of NDUFV1^{A341V}

### Materials and Methods

The molecules (ALV, 4-Hydroxy ALV and DMSO) were tested for their ability to restore the thermosensitive growth defect of the nuo-51^{A357V} mutant strain by drug drop test. The mutant mycelium, scratched from a 2 days culture on M2 plates at 28° C. was mixed in a FastPrep apparatus and spread on fresh square plates. 6 mm cellulose discs are then dropped and spotted with the various compounds at 10 mM in DMSO and plates were incubated at 33° C. Within 4 to 6 days a putative resumption of growth around filters (i.e. halo) is observed.

### Results

The results are shown in FIG. 3.

Alverine citrate (A: top corner left) and 4-Hydroxy Alverine (B: top corner right), the first metabolite of Alverine in its degradation pathway, rescue the growth of the nuo-51^{A357V} mutant strain without toxicity. The fact that the same effect is observed with both compounds shows that in the compound alverine citrate, it is alverine that is active.

### Example 3: Effect of ALV on the Growth Rate of the Nuo-51^{A357V} (NDUFV1), the ΔNuo-19 (NDUFS7) and the Nd2-Nd3 (ND) Mutant Strains

### Materials and Methods

The mutant strains were grown at 28° C., except for the thermosensitive nuo-51^{A357V} strain grown at 31.5° C. (as determined in example 1), 3-6 days in presence of various concentrations of ALV. Growth rates were calculated as described in Example 1.

Experiments were done at least in triplicates. Differences between treated vs untreated cultures (VEH) were evaluated according to the standard deviation (error bars).

### Results

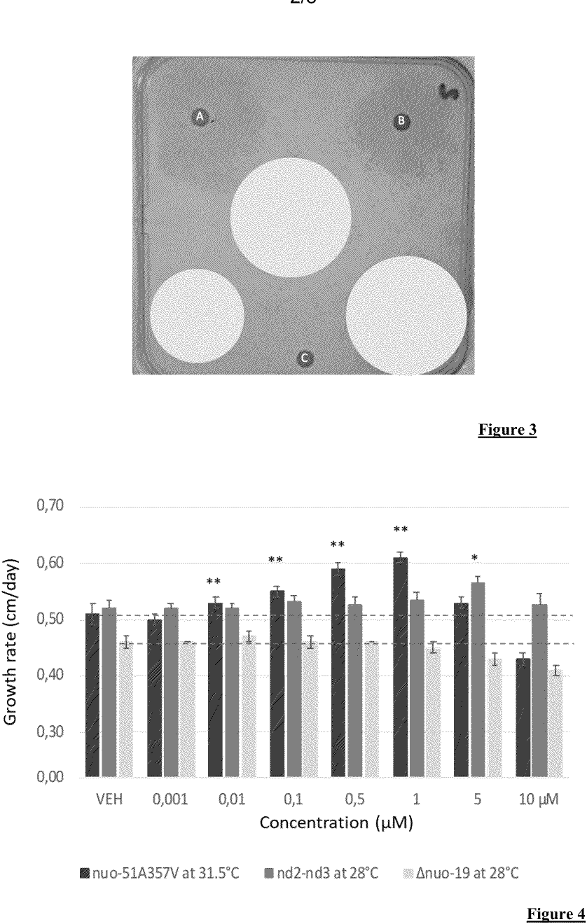

The data are shown in FIG. 4.

The nuo-51 ^{A357V} and the nd2-nd3 mutant strains exhibit an increased growth rate in the presence of ALV. On the contrary, ALV does not increase the growth rate of the Δnuo-19 strain (complete absence of functional complex I). The optimal ALV dose response determined from 5 independent experiments is 1 µM for the nuo-51 ^{A357V} strain and is used for further experiments. No toxicity can be detected under 10 µM.

### Example 4: Effect of ALV on the O2 Consumption Rate of Nuo-51^{A357V} (NDUFV1)

### Materials and Methods

The nuo-51 ^{A357V} mutant strain was grown at 31.5° C. in absence of ALV (VEH) or in the presence of 1 µM ALV (optimal concentration as determined in example 3) and 40 mg mycelium were introduced in an Oxytherm apparatus (Hansatech electrode) as described in Example 1.

Experiments were done at least five times and error bars represent the standard deviation. Differences between treated vs untreated cultures (VEH) were evaluated using the Pearson’s chi-square test, with significant p values < 0.05.

### Results

The results are represented in FIG. 5.

The respiration rate of the nuo-51 ^{A357V} mutant is significantly increased in the presence of 1 µM ALV.

### Example 5: Effect of ALV on Complex I Activity of Nuo-51^{A357V} (NDUFV1)

### Materials and Methods

The nuo-51 ^{A357V} mutant strain was grown at 31.5° C. in the presence (1 µM) or absence of ALV (VEH) and the rotenone sensitive-NADH dehydrogenase activity was determined on crude mitochondrial extracts as described in Example 1.

Experiments were performed at least five times and error bars represent the standard deviation. Differences between treated vs untreated cultures (VEH) were evaluated using the Pearson’s chi-square test, with significant p values < 0.05.

### Results

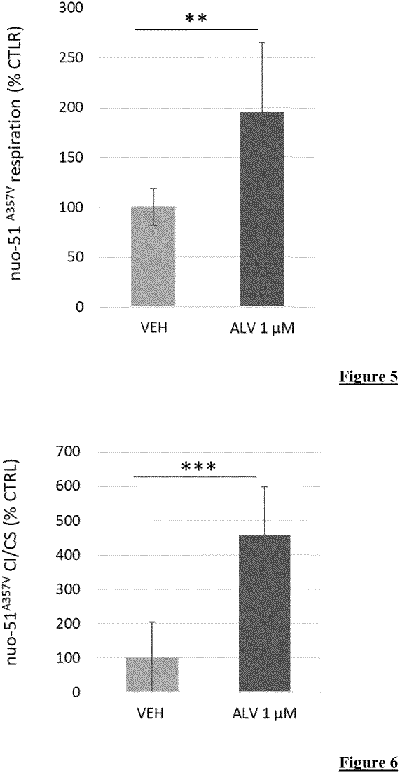

The results are represented in FIG. 6.

Complex I activity of the nuo-51 ^{A357V} mutant is significantly increased in the presence of 1 µM ALV.

### EXAMPLES 6 TO 7: CAENORHABDITIS ELEGANS MODEL

The C. elegans nematodes used in Examples 6 to 7 are the wild type BRISTOL N2 line from the Caenorhabditis Genetics Center (CGC) consortium. The mutant line LB25, carrying the nuo-1^{A352V} mutation (Δnuo-1, ex:nuo-1 (A352V)), mimicking the pathogenic mutation NDUFV1^{A341V} in human, was constructed by B. Lemire (Canada; Grad and Lemire, 2004).

### Example 6: Effect of ALV on C. Elegans Nuo-1^{A352V} (NDUFV1) Progeny

### Materials and Methods

Individual L4 animals (F0), allowed to develop from the L1 stage with or without ALV 1 µM, were transferred to separate wells in microplates, monitored daily during egg laying, and transferred to fresh wells to keep them separate from their progeny. 2-3 days after hatching, adults from the progeny (F1-adults) were counted.

The progeny of more than 30 FO-adults was recorded by experiment in three independent experiments and error bars represent the standard deviation. Differences between treated cells vs untreated cells were evaluated using the Student’s t-test with significant p values <0.05.

### Results

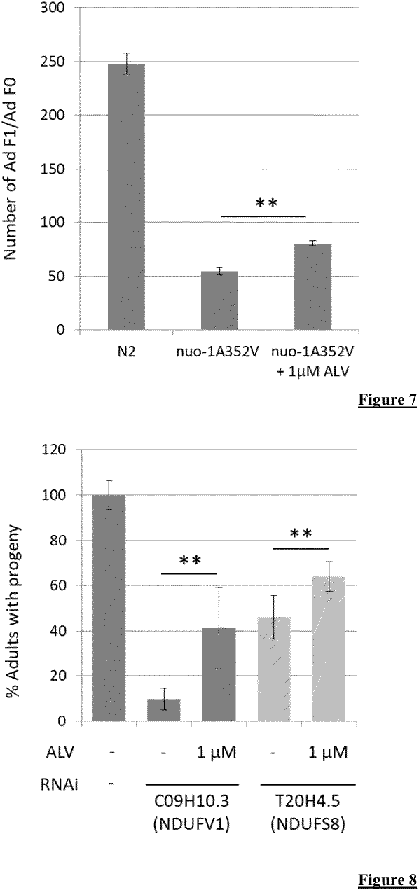

The results are represented in FIG. 7.

In the presence of 1uM ALV in the medium the adult progeny of nuo-1^{A352V} worms, i.e. the number of F1-adults obtained by F0-adult, is significantly increased (p=0.02).

### Example 7: Effect of ALV on the Progeny of Wild-Type Worms Subjected to NDUFV1 or NDUFS8 RNAi

### Materials and Methods

Decreased expression of NDUFV1 or NDUFS8 in wild-type worms (N2) was achieved by RNAi with the feeding method (Kamath RS and Ahringer J., 2003). Wild type worms were allowed to synchronously develop from the L1 stage to the L4 stage under the RNAi conditions and in presence or absence of 1 µM ALV. Individual L4 animals were then transferred to separate wells (same medium and RNAi conditions) in microplates and monitored within the 3 to 7 next days.

RNAi of NDUFV1 (CO9H10.3) and RNAi of NDUFS8 (T20H4.5) lead to few FO-adults able to give a larval progeny. The number of FO-adults able to give a larval progeny was thus monitored. The progeny of more than 30 FO-adults was recorded in three independent experiments (t-test, p=0.02). The efficiency of RNAi was determined by real-time qRT-PCR (20% remaining mRNA of NDUFV1 or NDUFS8 in the FO-adults under the RNAi conditions).

### Results

The results are represented in FIG. 8.

Under RNAi conditions leading to a down expression of NDUFV1 (part of the N module) or of NDUFS8 (part of the Q module), ALV 1 µM is able to increase their progeny.

### EXAMPLES 8 TO 12: HUMAN CELLULAR MODELS

As Alverine (ALV) was found to have a positive effect based on P. anserina mutant strains and worm mutants, it was then tested on human mutant cells derived from patients: skin fibroblast cells carrying complex I mutations (NDUFV1 mutation on c.1162+2A>C, c.1156C>T; (p.Arg386Cys) are used in examples 8 and 9.

### Example 8: Effect of ALV (Concentrations From 30 nM to 30 µM) on Cell Proliferation of NDUFV1 Fibroblast Mutant Cells Carrying Compound Heterozygous Mutations c.1162+2A>C and c.1156C>T (p.Arg386Cys)

### Materials and Methods

NDUFVI mutant fibroblasts were cultured in 24 well plates in low glucose medium (0.5 g/l) DMEM-F12 supplemented with 10% fetal bovine serum, 1% glutamine, as described in Leman et al. (2015). Growth proliferation and confluence were monitored in real time during 96 hours in wells seeded at the same cell density in an automated way using the CCD camera of the IncuCyte® live-cell analysis system device at 37° C. in presence of 5% CO2. Mutant cells were treated with different concentrations of ALV (30 nM to 30 µM) versus vehicle. Proliferation time-course revealed concentration-dependent treatment effects according to cell confluence.

### Results

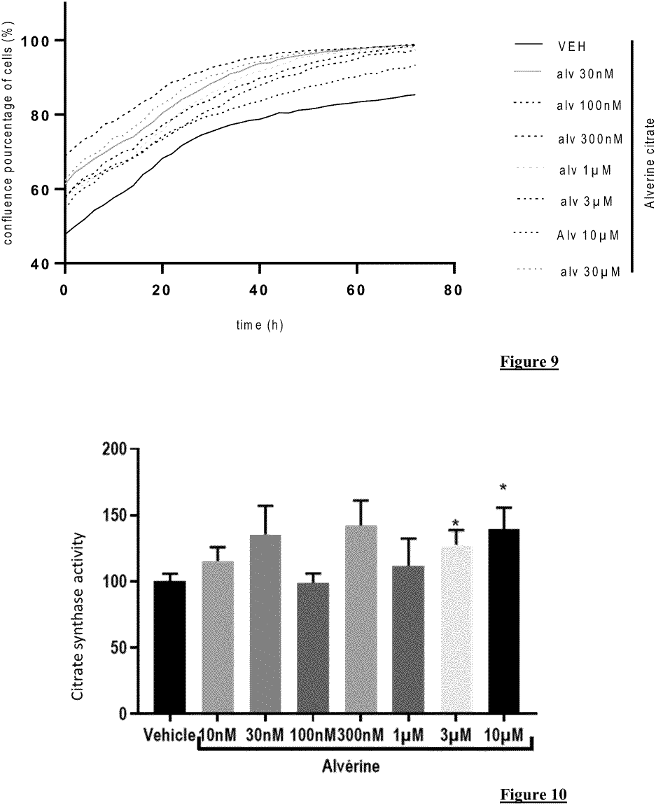

The results are represented in FIG. 9.

It reveals that cell proliferation of NDUFV1 mutant fibroblast cells carrying the compound heterozygous mutations c.1162+2A>C and c.1156C>T (p.Arg386Cys) exposed to different ALV drug concentrations (from 30 nM to 30 µM) was reduced at 10 µM and 30 µM concentrations of ALV compared to Vehicle.

### Example 9: Determination of the Active ALV Concentration on Complex I Enzyme Activity on NDUFV1 Fibroblast Mutant Cells Carrying Compound Heterozygous Mutations c1162+2A>C, and c1156C>T (p.Arg386Cys)

### Materials and Methods

The complex I mutant cells, carrying the NDUFV1 compound heterozygous mutations responsible for complex I deficiency, were cultured in standard DMEM high glucose media (4.5 g/L) or in low glucose (0.5 g/L), supplemented with 10% fetal bovine serum, 1% glutamine and 50 µg/ml uridine at 37° C. in presence of 5% CO₂ as described elsewhere (Leman et al., 2015). To optimize the effect of drug concentrations, cells were shifted to low glucose-medium 0.5 g/l (to force the cells to rely on OXPHOS rather than glycolysis) supplemented with various concentrations of 10 nM to 10 µM of Alverine or of the vehicle (DMSO) during 48 hours.

Complex I enzyme activity was measured at 37° C. on an UVmc2 spectrophotometer (SAFAS) as described (Leman et al., 2015; Desquiret-Dumas et al., 2012). For complex I enzyme activity, 0.5 million of cells were sonicated (6 cycles of 5 seconds) then incubated at 37° C. in the reaction medium (KH₂PO₄ 100 mM, pH 7.4, KCN 1 mM, NaN₃ 2 mM, BSA 1 mg/ml, ubiquinone-1 0.1 mM and DCPIP 0.075 mM). The reaction was started by adding 0.15 mM NADH and the disappearing rate of DCPIP was measured at 600 nm for 2 minutes. The unspecific activity was determined in the presence of rotenone (5 µM).

The enzymatic activity of complex I was normalized with respect to citrate synthase (CS) activity which is considered as a marker of mitochondrial mass. Citrate synthase activity was measured by adding 0.1 million of cells to a prewarmed reaction mix composed of 5,5′-Dithiobis 2-nitrobenzoic acid (DTNB) 0.15 mM, oxaloacetic acid 0.5 mM, acetyl coA 0.3 mM, triton X100 0.1%) and the appearing rate of CoA-SH at 412 nm was assessed following the DTNB reduction.

Experiments were done at least in triplicates and error bars represent the standard deviation. Differences between treated cells vs untreated cells (DMSO) were evaluated using the Student’s t-test with significant p values <0.05.

### Results

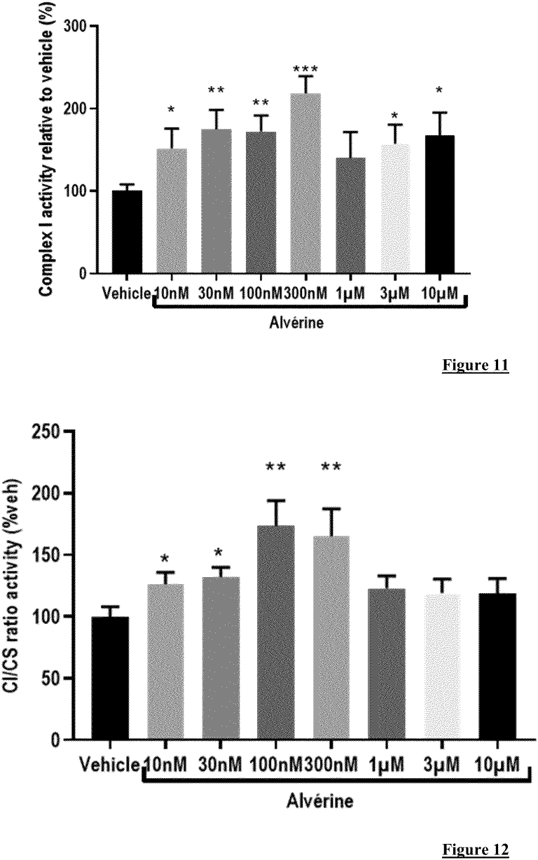

The results are represented in FIGS. 10 to 12. The asterisks (*, ** or ***) indicate a statistically significant difference versus the untreated cells (Vehicle).

The data reveal that ALV, at concentrations of 100 nM and 300 nM, has a beneficial effect on complex I enzyme activity of NDUFV1 mutant cells, and that ALV concentrations of 300 nM up to 10 µM have no deleterious effect on the activity of complex I in mutant cells.

### Example 10: Effect of ALV (Concentrations From 30 nM to 10 µM) on Cell Growth Proliferation of ND6 Fibroblast Mutant Cell Line Carrying the m.14484T>C (p.Met64Val) in MT-ND6 Gene

Complex I is encoded by the nuclear and mitochondrial genomes. As Alv was shown to have a positive effect on fibroblast carrying a mutation in NDUFV1, a nuclear-encoded subunit, it was then tested on fibroblast complex I deficient cells carrying the homoplasmic m.14484T>C (p.Met64Val) mitochondrial DNA mutation, shown to be responsible for LHON disease.

### Materials and Methods

ND6 mutant fibroblast cells were cultured in 24 well plates in low glucose medium (0.5 g/l) DMEM-F12 supplemented with 10% fetal bovine serum, 1% glutamine as described in Leman et al. (2015). Growth proliferation and confluence were monitored in real time during 96 hours in wells seeded at the same cell density in an automated way using the CCD camera of the IncuCyte® live-cell analysis system device, at 37° C. in presence of 5% CO2. Mutant cells were treated with different concentrations of ALV (30 nM to 10 µM) versus vehicle. Proliferation time-course revealed concentration-dependent treatment effects according to cell confluence.

### Results

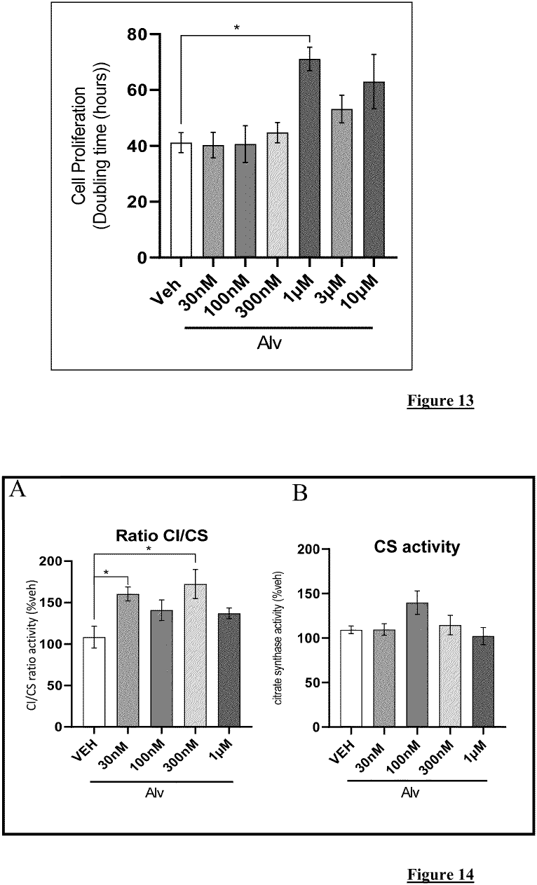

The results are represented in FIG. 13.

It reveals that cell proliferation of ND6 mutant fibroblast cells carrying the homoplasmic m.14484T>C (p.Met64Val) mitochondrial DNA mutation exposed to different ALV drug concentrations (from 30 nM to 10 µM) was unchanged at the concentrations of 30, 100 and 300 nM. However, a significant increase in doubling time (i.e. a reduction of the cell proliferation) was observed at a concentration ≥ 1 µM ALV compared to Vehicle.

### Example 11: Measurement of Complex I Enzymatic Activity for ND6 Mutant Cell Line Carrying the Homoplasmic m.14484T>C (p.Met64Val) Mitochondrial DNA Mutation After ALV Treatment

### Materials and Methods

The complex I mutant cells, carrying the homoplasmic m.14484T>C (p.Met64Val) mitochondrial DNA mutation responsible for complex I deficiency, were cultured in standard DMEM high glucose media (4.5 g/L) or in low glucose (0.5 g/L), supplemented with 10% fetal bovine serum, 1% glutamine and 50 µg/ml uridine at 37° C. in presence of 5% CO₂ as described elsewhere (Leman et al., 2015). To optimize the effect of drug concentrations, cells were shifted to low glucose-medium 0.5 g/l (to force the cells to rely on OXPHOS rather than glycolysis) supplemented with various concentrations of 30 nM to 10 µM of Alverine or of the vehicle (DMSO) during 48 hours.

Complex I enzyme activity was measured at 37° C. on an UVmc2 spectrophotometer (SAFAS) as described (Leman et al., 2015; Desquiret-Dumas et al., 2012). For complex I enzyme activity, 0.5 million of cells were sonicated (6 cycles of 5 seconds) then incubated at 37° C. in the reaction medium (KH2PO4 100 mM, pH 7.4, KCN 1 mM, NaN3 2 mM, BSA 1 mg/ml, ubiquinone-1 0.1 mM and DCPIP 0.075 mM). The reaction was started by adding 0.15 mM NADH and the disappearing rate of DCPIP was measured at 600 nm for 2 minutes. The unspecific activity was determined in the presence of rotenone (5 µM).

The enzymatic activity of complex I was normalized with respect to citrate synthase (CS) activity which is considered as a marker of mitochondrial mass. Citrate synthase activity was measured by adding 0.1 million of cells to a prewarmed reaction mix composed of 5,5′-Dithiobis 2-nitrobenzoic acid (DTNB) 0.15 mM, oxaloacetic acid 0.5 mM, acetyl coA 0.3 mM, triton X100 0.1%) and the appearing rate of CoA-SH at 412 nm was assessed following the DTNB reduction.

Experiments were done at least in triplicates and error bars represent the standard deviation. Differences between treated cells vs untreated cells (DMSO) were evaluated using the Student’s t-test with significant p values <0.05.

### Results

The results are represented in FIG. 14. The asterisks (*) indicate a statistically significant difference versus the untreated cells (Vehicle).

The data reveal that ALV, at concentrations of 30 nM and 300 nM, has a beneficial effect on complex Ienzyme activity of ND6 mutant cells normalized to citrate synthase activity.

### Example 12: Determination of Lactate Production in NDUFV1 and ND6 Mutant Cell Lines Exposed to Different Concentrations of ALV From 30 nM to 10 µM

The mutant cell lines have a preferential anaerobic glycolytic metabolism compared to control cells. The determination of lactate production was assessed after a 48 h exposure in the presence of different concentrations of Alv from 30 nM to 10 µM.

### Materials and Methods

A total of 15,000 cells/well were plated in 96-well plates and grown at 37° C. in the presence of 5% CO2. After 24 h, the cells were treated with different concentrations of Alv from 30 nM to 10 µM. After 48 h, supernatants were harvested and lactates were assayed using a lactate determination kit according to the manufacturer (Abcam kit ab65330). Lactate concentration was measured with a microplate spectrophotometer (CLARIOstar apparatus, BMG Labtech).

### Results

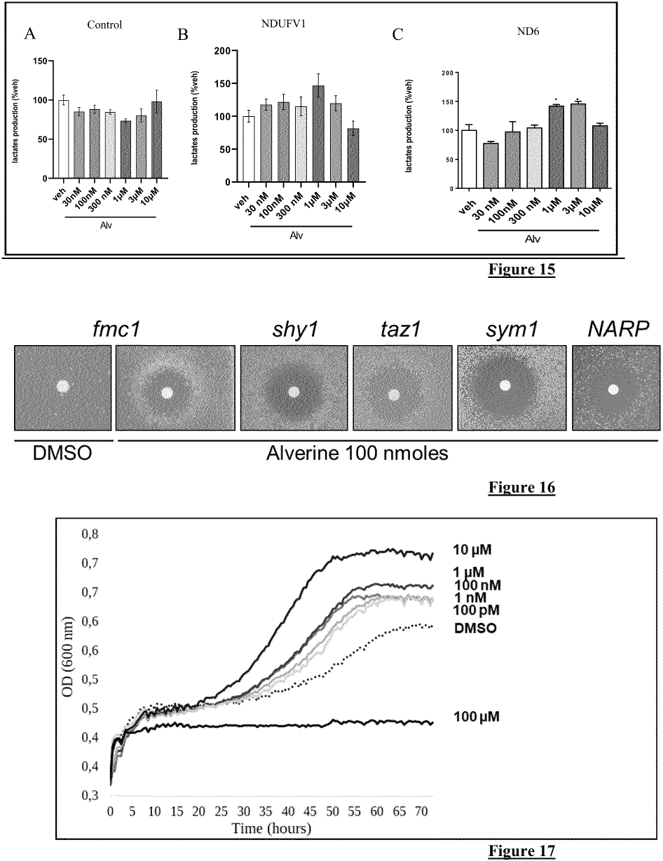

The results are represented in FIG. 15.

Alv did not alter lactate production for the control fibroblast cell line (FIG. 15A) and for the NDUFV1 cell line (FIG. 15B). Lactate production for the ND6 cell line (FIG. 15C) was significantly increased for concentrations above 1 µM and 3 µM compared to vehicle.

### EXAMPLES 13 TO 14: EFFICACY OF ALVERINE ON OTHER MITOCHONDRIAL YEAST MUTANTS

### Example 13: Effect of Alverine on Growth of Various Mutant Yeast Strains

### Materials and Methods

### Yeast Mutant Strains

taz1Δ yeast strain was constructed by replacing the open reading frame of TAZ1 by that of TRP1 in the W303-1A strain (MATa ade2-1 ura3-1 his311, 15 trp1-1 leu2-3,112 can1-100) (de Taffin de Tilques, M. et al., 2018).

sym1Δ yeast strain was constructed by replacing the open reading frame of SYM1 by that of kanMX6 in the W303-1A strain (MATa ade2-1 ura3-1 his311, 15 trpl-1 leu2-3,112 can1-100). fmc1 MC6 MATa ade2-1 his3-11,15 trp1-1 leu2-3,112 ura3-1 fmcl::HIS3 [Δi ER OR] (Schwimmer, C. et al., 2005).

NARP MR14 MATa ade2-1 his3-11,15 trpl-1 leu2-3,112 ura3-1 CAN1 arg8::HIS3 ρ+ atp6-L183R (Rak, M. et al., 2007).

shy1Δ yeast strain was constructed by replacing the open reading frame of SHY1 by that of HIS in the W303-1A strain (MATa ade2-1 ura3-1 his311, 15 trp1-1 leu2-3,112 can1-100) (Barrientos, A. et al., 2002).

200 µL of the various yeast mutant strain grown in liquid YPD rich fermentable medium (1% Yeast extract, 0.5% Bacto Peptone, 2% Glucose) at 0.5 OD₆₀₀ were spread on agar-based solid respiratory medium: either YPG (1% Yeast Extract, 0.5% Bacto Peptone, 2% glycerol) for fmc1, shy1 and NARP mutants or YPE (1% Yeast extract, 0.5% Bacto Peptone, 2% ethanol) for taz1 and sym1 mutants. Small sterile filters were then placed on the agar surface and 100 nmoles of Alverine (ALV) were added to the filters. The plates were then incubated at 28° C. for shy1 mutant or 36° C. for fmc1, tazl, sym1 and NARP mutants for 4-5 days and then scanned. DMSO, the compound vehicle is used as a negative control.

### Results

As shown on FIG. 16, halo of enhanced growth is observed around the filters where ALV has been spotted indicating that ALV is effective on all the tested yeast models of mitochondrial diseases.

### Example 14: Determination of the Active Range of Concentration of ALV Leading to Suppression of the Respiratory Growth Defect of the Taz1 Mutant Yeast Strain

### Materials and Methods

Exponentially growing cells were inoculated in fresh non-fermentable YPG media supplemented, or not, with increasing ALV concentrations. These assays were performed in 96-well plates in the Bioscreen device. Cell density was followed over time in order to determine both the optimal concentrations of ALV as for its ability to suppress respiratory growth defect and the concentration at which it displays toxicity. The taz1 cells have been grown for 70 h in liquid medium containing 2% glycerol as carbon source and increasing concentrations of ALV (100 pM to 100 µM).

### Results

As shown on FIG. 17, an increased respiratory growth is observed from 100 pM to 10 µM indicating that the therapeutic window is quite large. A toxicity has been found from 100 µM.

### REFERENCES

Barrientos A, Korr D, Tzagoloff A. Shylp is necessary for full expression of mitochondrial COX1 in the yeast model of Leigh’s syndrome. EMBO J. 2002 Jan 15;21(1-2):43-52.

Barth PG, Van den Bogert C, Bolhuis PA, Scholte HR, van Gennip AH, Schutgens RB, Ketel AG. X-linked cardioskeletal myopathy and neutropenia (Barth syndrome): respiratory-chain abnormalities in cultured fibroblasts. J Inherit Metab Dis. 1996;19(2):157-60.

Desquiret-Dumas V, Gueguen N, Barth M, Chevrollier A, Hancock S, Wallace DC, Amati-Bonneau P, Henrion D, Bonneau D, Reynier P, Procaccio V. Metabolically induced heteroplasmy shifting and L-arginine treatment reduce the energetic defect in a neuronal-like model of MELAS (2012) Biochimica et Biophysica Acta Molecular Basis of Disease. 1822(6):1019-29.

De Taffin de Tilques M, Lasserre JP, Godard F, Sardin E, Bouhier M, Le Guedard M, Kucharczyk R, Petit PX, Testet E, di Rago JP, Tribouillard-Tanvier D. Decreasing cytosolic translation is beneficial to yeast and human Tafazzin-deficient cells. Microb Cell. 2018 Feb 18;5(5):220-232.

El-Khoury R, Sellem CH, Coppin E, Boivin A, Maas MF, Debuchy R, Sainsard-Chanet A. Gene deletion and allelic replacement in the filamentous fungus Podospora anserina. (2008) Curr Genet. 53:249-58.

Gorman GS, Schaefer AM, Ng Y, Gomez N, Blakely EL, Alston CL, Feeney C, Horvath R, Yu-Wai-Man P, Chinnery PF, Taylor RW, Turnbull DM, McFarland R. Prevalence of nuclear and mitochondrial DNA mutations related to adult mitochondrial disease. Ann Neurol. 2015 May; 77(5):753-9.

Grad LI and Lemire BD. (2004) Mitochondrial complex I mutations in Caenorhabditis elegans produce cytochrome c oxidase deficiency, oxidative stress and vitamin-responsive lactic acidosis. Hum. Mol. Genet. 13(3):303-14.

Johns DR, Neufeld MJ, Park RD. An ND-6 mitochondrial DNA mutation associated with Leber hereditary optic neuropathy. Biochem Biophys Res Commun. 1992 Sep 30;187(3):1551-7.

Kamath RS and Ahringer J. (2003) Genome-wide RNAi screening in Caenorhabditis elegans. Methods 30(4) :313-21.

Leman G, Gueguen N, Desquiret-Dumas V, Kane MS, Wettervald C, Chupin S, Chevrollier A, Lebre AS, Bonnefont JP, Barth M, Amati-Bonneau P, Verny C, Henrion D, Bonneau D, Reynier P, Procaccio V. Assembly defects induce oxidative stress in inherited mitochondrial complex I deficiency. Int J Biochem Cell Biol. 2015 May 27; 65:91-103.

Maas MF, Sellem CH, Hoekstra RF, Debets AJ, Sainsard-Chanet A. (2007) Integration of a pAL2-1 homologous mitochondrial plasmid associated with life span extension in Podospora anserina. Fungal Genet Biol.; 44(7):659-71.

Maas MF, Sellem CH, Krause F, Dencher NA, Sainsard-Chanet A. (2010) Molecular gene therapy: overexpression of the alternative NADH dehydrogenase NDI1 restores overall physiology in a fungal model of respiratory complex I deficiency. J Mol Biol.; 399(1):31-40.

Procaccio V, Wallace D.C. Late-onset Leigh syndrome in a patient with mitochondrial complex I NDUFS8 mutations. Neurology (2004) 62:1899.

Rak M, Tetaud E, Duvezin-Caubet S, Ezkurdia N, Bietenhader M, Rytka J, di Rago JP. A yeast model of the neurogenic ataxia retinitis pigmentosa (NARP) T8993G mutation in the mitochondrial ATP synthase-6 gene. J Biol Chem. 2007 Nov 23;282(47):34039-47.

Sarzi E, Brown M, Lebon S, Chretien D, Munnich A, Rotig A, Procaccio V. A novel recurrent mtDNA mutation in ND3 gene causing Leigh syndrome and dystonia. American Journal of Medical Genetics (2007) 143: 33-41.

Schon EA, Santra S, Pallotti F, Girvin ME. Pathogenesis of primary defects in mitochondrial ATP synthesis. Semin Cell Dev Biol. 2001 Dec;12(6):441-8.

Schuelke M, et al. Mutant NDUFV1 subunit of mitochondrial complex I causes leukodystrophy and myoclonic epilepsy. Nat Genet. 1999; 21(3):260-61.

Schwimmer C, Lefebvre-Legendre L, Rak M, Devin A, Slonimski PP, di Rago JP, Rigoulet M. Increasing mitochondrial substrate-level phosphorylation can rescue respiratory growth of an ATP synthase-deficient yeast. J Biol Chem. 2005 Sep 2;280(35):30751-9.

Spinazzola A, Viscomi C, Fernandez-Vizarra E, Carrara F, D′Adamo P, Calvo S, Marsano RM, Donnini C, Weiher H, Strisciuglio P, Parini R, Sarzi E, Chan A, DiMauro S, Rötig A, Gasparini P, Ferrero I, Mootha VK, Tiranti V, Zeviani M. MPV17 encodes an inner mitochondrial membrane protein and is mutated in infantile hepatic mitochondrial DNA depletion. Nat Genet. 2006 May;38(5):570-5.

Wang, K., Takahashi, Y., Gao, Z.-L., Wang, G.-X., Chen, X.-W., Goto, J., Lou, J.-N., Tsuji, S. Mitochondrial ND3 as the novel causative gene for Leber hereditary optic neuropathy and dystonia. Neurogenetics. 10: 337-345, 2009.

1-15. (canceled)

16. A method of treating a disease associated with mitochondrial dysfunction in a subject in need thereof, the method comprising: administering to the subject a pharmaceutical composition comprising alverine or a derivative thereof.

17. The method of claim 16, wherein the composition comprises alverine citrate or 4-hydroxy alverine.

18. The method of claim 16, wherein the disease is a mitochondrial respiratory chain disease.

19. The method of claim 16, wherein the disease is associated with mitochondrial complex I deficiency.

20. The method of claim 16, wherein the disease is a genetic disease.

21. The method of claim 20, wherein the genetic disease comprises at least one gene defect in at least one of the following genes: MTND1, MTND2, MTND3, MTND4, MTND5, MTND6, MTND4L, NDUFA1, NDUFA2, NDUFA3, NDUFA4, NDUFA5, NDUFA6, NDUFA7, NDUFA8, NDUFA9, NDUFA10, NDUFA11, NDUFA12, NDUFA13, NDUFAB1, NDUFB1, NDUFB2, NDUFB3, NDUFB4, NDUFB5, NDUFB6, NDUFB7, NDUFB8, NDUFB9, NDUFB10, NDUFB11, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3, NDUFAF1, NDUFAF2, NDUFAF3, NDUFAF4, NDUFAF5, NDUFAF6, NDUFAF7, NDUFAF8, NUBPL, ACAD9, TMEM126B, FOXRED1, ECSIT, AIF, TIMMDC1, MTTL1, ATP6, TAZ, SURF1, POLG, MPV17, OPA1, COA6, or BCS1L.

22. The method of claim 21, wherein the genetic disease comprises at least one gene defect in MTND3, MTND6, NDUFV1, NDUFS8, ATP6, TAZ, SURF1, or MPV17.

23. The method of claim 16, wherein the disease is MELAS syndrome, maternally inherited myopathy and cardiomyopathy, NARP syndrome, Leigh syndrome, Barth syndrome, Mitochondrial DNA Depletion Syndrome, Mitochondrial DNA Depletion Syndrome 4A (Alpers Type), Mitochondrial DNA Depletion Syndrome 4B (MNGIE Type), Mitochondrial recessive ataxia syndrome, Sensory Ataxic Neuropathy Dysarthria and Ophthalmoplegia, Spinocerebellar Ataxia with Epilepsy, Progressive External Ophthalmoplegia, Mitochondrial DNA depletion syndrome-6, Navajo neuropathy, Behr Syndrome, Mitochondrial DNA Depletion Syndrome 14, infantile cardioencephalomyopathy due to cytochrome c oxidase deficiency (COA6 mutations), Mitochondrial Complex III Deficiency Nuclear Type 1, GRACILE Syndrome, Leber’s optic hereditary neuropathy, or Bjornstad Syndrome.

24. The method of claim 23, wherein the disease is NARP syndrome, Barth syndrome, Mitochondrial DNA Depletion Syndrome, Leigh syndrome, Leber’s optic hereditary neuropathy, or MELAS syndrome.

25. The method of claim 16, wherein the disease is Alzheimer’s disease, Parkinson’s disease, Huntington’s disease, amyotrophic lateral sclerosis (Lou Gehrig’s disease), Friedreich’s ataxia, cardiovascular diseases, atherosclerosis, diabetes, metabolic syndrome, autoimmune diseases, multiple sclerosis, systemic lupus erythematosus, type I diabetes, neurobehavioral diseases, psychiatric diseases, autism spectrum disorders, schizophrenia, bipolar disorders, mood disorders, gastrointestinal disorders, fatiguing illnesses, chronic fatigue syndrome, Gulf War illnesses, musculoskeletal diseases, fibromyalgia, skeletal muscle hypertrophy/atrophy, muscular dystrophies, cancer, or chronic infections.

26. The method of claim 16, wherein the composition is associated with other treatments for the same disease.

27. The method of claim 16, wherein the composition comprises another compound for treating the same disease.

28. The method of claim 16, wherein the composition is administered orally.

29. The method of claim 16, wherein the composition is administered daily.

30. The method of claim 16, wherein the composition is in a solid form.

31. The method of claim 16, wherein the composition is in the form of a tablet.

32. The method of claim 16, wherein the composition comprises 60 mg of alverine or a derivative thereof.
